(19) United States
(12) Patent Application Publication
(10) Pub. No.: US 2026/0338723 A1
(43) Pub. Date: Oct. 29, 2026

(54) PHYLLOSILICATE CHEMICAL GAS SENSOR

(71) Applicant: University of Notre Dame du Lac, South Bend, IN (US)

(72) Inventors: JoAnna Milam-Guerrero, South Bend, IN (US); Nosang Myung, South Bend, IN (US); Faraj Al-badani, South Bend, IN (US)

(21) Appl. No.: 19/120,592

(22) PCT Filed: Oct. 13, 2023
(86) PCT No.: PCT/US2023/076830

§ 371 Date: Apr. 11, 2025

Related U.S. Application Data
(60) Provisional application No. 63/379,301, filed on Oct. 13, 2022.

(51) Int. Cl. G01N 27/12; G01N 33/00

(52) U.S. Cl. CPC G01N 27/125; G01N 33/0031; G01N 33/004; G01N 33/0044; G01N 33/0047; G01N 33/0054

(57) ABSTRACT

A gas sensor device includes a sensor array and a controller. The sensor array includes a plurality of sensors and each of the plurality of sensors includes a sensing element, a heating element, and a lighting element. The sensing element includes an electrode pair and a sensing material electrically coupled to the electrode pair. The sensing material includes a phyllosilicate and is configured to detect a presence of a target analyte such that an electrical property of the sensing material changes in response to detection of the target analyte by the sensing material. The controller is communicatively coupled to the sensor array and configured to adjust the heating element to cause the heating element to influence a temperature of the sensing element and configured to adjust the lighting element to cause the lighting element to influence an illumination of the sensing element.

### CROSS-REFERENCE TO RELATED APPLICATION

This application is a non-provisional application claiming priority from U.S. Provisional Application Ser. No. 63/379,301, filed Oct. 13, 2022, entitled “Phyllosilicate Based Chemical Gas Sensor” and incorporated herein by reference in its entirety.

### STATEMENT REGARDING FEDERALLY SPONSORED RESEARCH OR DEVELOPMENT

This invention was made with government support under grant N64267-19-C-0024 awarded by the Naval Sea Systems Command and grant NB18-21-27 awarded by US Air Force Research Laboratory (AFRL). The government has certain rights in the invention.

### TECHNICAL FIELD

The present description relates generally to detecting analytes using high performance sensing materials and more particularly to an electrical detection gas sensor (e.g., chemiresistor, chemi-capacitor, impedimetric sensor) such as a phyllosilicate chemical gas sensor.

### BACKGROUND

Certain gas sensors can detect environmental compounds, such as inorganic gasses, volatile organic compounds (VOCs) and chemical warfare agents (CWAs). In addition, some gas sensors can also be used for environmental monitoring or for providing information about manufacturing processing conditions. However, it is often infeasible to dedicate time to collect samples of gasses or other materials from the environment for laboratory analysis. Therefore, it is desirable to have an automated device that can check the presence of certain targeted analytes or other materials.

In a similar disclosure, U.S. Application Ser. No. 63/268,814 discloses an intelligent electronic nose system to identify different analytes. The contents of U.S. Ser. No. 63/268,814 are incorporated herein by reference in its entirety.

### BRIEF DESCRIPTION OF THE DRAWINGS

FIG. 1 is a diagram of an example detection device for detecting various substances of interest in accordance with the various examples disclosed herein.

FIG. 2 is a is a flow chart illustrating an example method of preparing a sensing material in accordance with the various examples disclosed herein.

FIG. 3 is a flow chart illustrating an example method of preparing a detection device in accordance with the various examples disclosed herein.

FIG. 4 is a flow chart illustrating an example method of detecting various target analytes of interest in accordance with the various examples disclosed herein.

### DETAILED DESCRIPTION

The following disclosure of example methods and apparatus is not intended to limit the scope of the description to the precise form or forms detailed herein. Instead, the following disclosure is intended to be illustrative so that others may follow its teachings.

There exist several methods of gas sensing (e. g, electrochemical, optical, mass sensitive, electrical including chemiresistive, chemicapacitive, and impedimetric, etc.). In general, electrical gas sensors provide low-cost sensing with high response/recovery times and simple signal acquisition processes that have led to more portable versions with increasing functionality and sensitivity. Electrical semiconductor gas sensors (chemiresistors) are the most common commercially available sensor type where, upon exposure to analytes, a subsequent resistance change occurs from oxidative/reductive interactions. The type of resistance response (increase or decrease in resistance) depends on whether the sensing material is an n-type or p-type semiconducting material which is a function of the charge carrier being either electrons or holes, respectively.

In at least one example of the following disclosure, transitional metal phyllosilicates or their derivatives (e.g., heat treated phyllosilicates) are utilized as gas sensing materials that are capable of sensing inorganic and volatile organic compound (VOC) gasses. Phyllosilicates, or clays, are layered materials that are characterized by two-dimensional sheets of silicon-oxygen (SiO₄) tetrahedra and aluminum-oxygen (AlO₄) octahedra. Phyllosilicates can be separated into two subgroups, 1:1 or 1:2, according to their silicon (Si):metal ratio. In the 1:1 subgroup, the clay comprises one tetrahedral and one octahedral group in each layer. In the following disclosure, 1:1 clays are delaminated and leveraged to behave as pseudo-one-dimensional layers with a catalytic metal oxide layer connected to a non-conductive silicon layer.

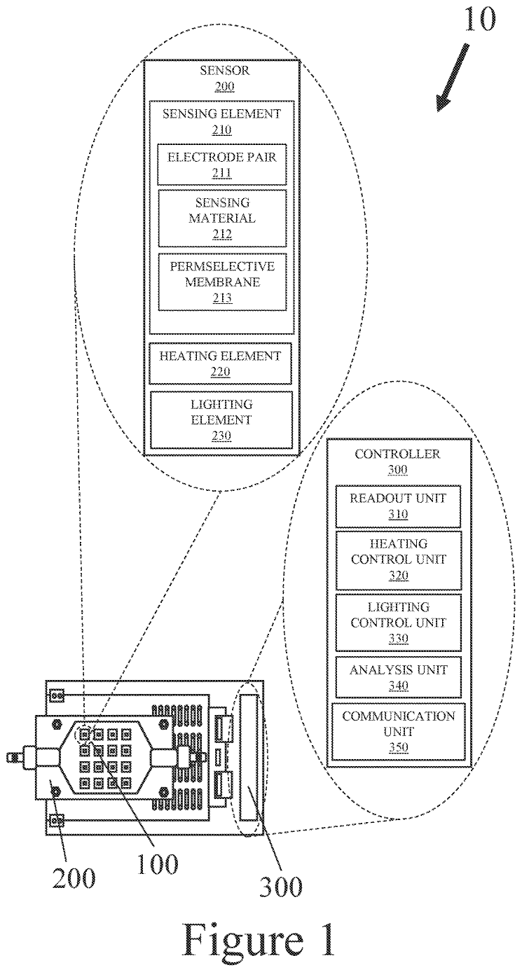

Referring now to the drawings, FIG. 1 is a diagram of an example detection device 10 for detecting gaseous target analytes. As shown in FIG. 1, the example detection device 10 includes a sensor array 100 containing one or more sensors 200 and a controller 300. The sensor 200 of the example detection device 10 includes a sensing element 210, a heating element 220, and a lighting element 230. The sensing element 210 of the sensor 200 may include an electrode pair 211, a sensing material 212, and a permselective membrane 213. The sensing material 212 electrically bridges the electrode pair 211.

The sensing material 212 may be any suitable material for use in an electrical sensor (e.g., chemiresistor, chemicapacitor, impedimetric sensor) such that the sensing material 212 changes its electrical properties (e.g., resistance, capacitance, or impedance) in response to changes in the nearby chemical environment (e.g., direct chemical interaction between the sensing material 212 and a target analyte, or substance of interest, in the chemical environment). For instance, the example sensing material 212 and the analyte may interact by physical or chemical adsorption and desorption, chemical reaction (e.g., catalytic oxidation or reduction), molecular recognition (e.g., covalent bonding, hydrogen bonding, and Val der Waals interaction), or other suitable interaction. Based on the changes caused by this molecular interaction, an output of the sensing material 212 can be used to evaluate the presence (or lack thereof) of a target analyte in the air or other ambient atmosphere.

As used herein, target analyte may refer to any substance whose chemical constituents are being identified and measured. The substance may be a chemical substance (e.g., ammonia, hydrogen sulfide, etc.), a gas, a vapor, a fume, an odor, a smell, or other suitable substance, for example. As such, while reference in this disclosure may be made to the analyte as a chemical or hazardous material, the disclosure should not be read as limited to such and should instead be read as applicable to any suitable sensible substance contained in a gaseous environment. Furthermore, while reference is made primarily to a single analyte being sensed (e.g., “target analyte”), this disclosure should not be read as limited to the sensing of a single target analyte and should instead be read as applicable to the sensing of one or more target analytes at a time.

In the present example, the sensing material 212 is configured to detect a target analyte. In some examples, the target analyte is at least one of an inorganic compound, a volatile organic compound, or any combinations thereof. The inorganic compound may be ammonia, nitric oxide, nitrogen dioxide hydrogen sulfide or carbon monoxide. Further, the volatile organic compound may be toluene, ethanol, acetone or benzene.

The example sensing material 212 comprises a phyllosilicate or phyllosilicate derivative. In other examples, the phyllosilicate may include a transition metal and the transition metal may include at least one of copper, cobalt, iron, nickel, manganese, zinc, titanium, tin, tungsten, or any combinations thereof. It will be understood that in various instances, the sensing material may be a nanomaterial.

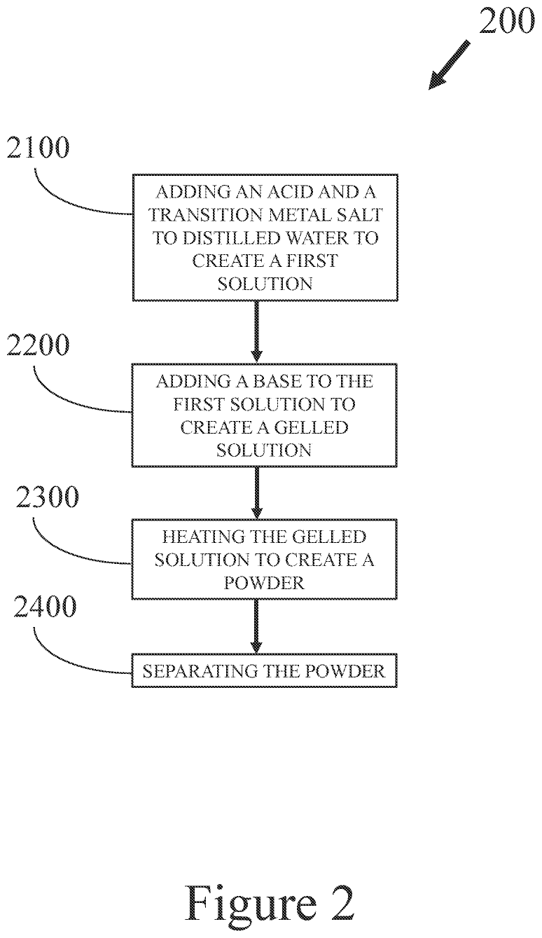

As shown in FIG. 2, the sensing material 212 may be prepared by an example method 200. The example method 200 includes: an adding step 2100, a gelling step 2200, a heating step 2300, and a separating step 2400. More precisely, in this example, the adding step 2100 includes adding an acid and a transition metal salt to distilled water to create a first solution. In some examples, the acid is silicic acid (H₂SiO₃), while in some examples, the transition metal salt is a nitrate.

Once the adding step 2100 is completed, the example gelling step 2200 includes adding a base to the first solution of the step 2100 to create a gelled solution. In some examples, the base is sodium hydroxide (NaOH). While any suitable method of gelling may be employed, in this example, the base is added drop-wise to the first solution of the step 2100 and after the addition of the base, the solution may be allowed to sit for a period of time, such as for instance, 72 hours, to create a gelled solution.

After the example gelling step 2200, the method 200 may utilize the heating step 2300, which in this instance, includes heating the gelled solution of the step 2200 to create a powder. The powder may be a nanopowder or a micropowder. In the present example, the heating occurs at a temperature in a range of from 150° C. to 250 °C., while other ranges may be suitable utilized as desired. In at least one example, the heating occurs at a temperature of around 200° C., which may be considered an optimized temperature. As noted, in some other examples, the heating may occur for any suitable time period, including for 50 hours.

Once the heating step 2300 is sufficiently progressed, the separating step 2400 may include any suitable separation process, such as at least one of centrifugation process, washing process, drying process, or any combination thereof. In the present example, the separating step 2400 may include a drying step at a temperature of around 80° C., while a suitable drying process may occur at a temperature in a range of from 60° C. to 100° C. It will be further appreciated that while any suitable time frame for drying may be utilized, in the present examples, the drying occurs for a time of from 1 hour to 12 hours.

As disclosed herein, the sensing material 212 is a composite material comprising two or more materials combined together. In some examples, however, the sensing material 212 may include a permselective membrane 213. The permselective membrane 213 comprises a membrane that allows only some substances (e.g., target analyte) to pass through the membrane and interact with the sensing material 212 while blocking other molecules from interacting with the sensing material 212. In this way, the permselective membrane 213 is designed to at least partially control to which analyte(s) the sensing element 210 is sensitive. The permselective membrane may be an organic (e.g., polymer with ionic side groups (e.g., ion-exchange resins)) or a covalent organic framework (COF) or crystalline micro/mesoporous hybrid materials (e.g., metal organic framework (MOF)). In some examples, the permselective membrane 213 is omitted.

Although reference is made throughout to a single sensing material 212 and/or a single permselective membrane 213, this disclosure should not be read as limited to inclusion of a single sensing material 212 and a single permselective membrane 213 but should instead be read as applicable to embodiments in which multiple sensing materials 212 and multiple permselective membranes 213 are included in the sensing element. Furthermore, the various sensors 200 comprising the sensor array 100 may each employ different sensing materials 212 and/or different permselective membranes 213.

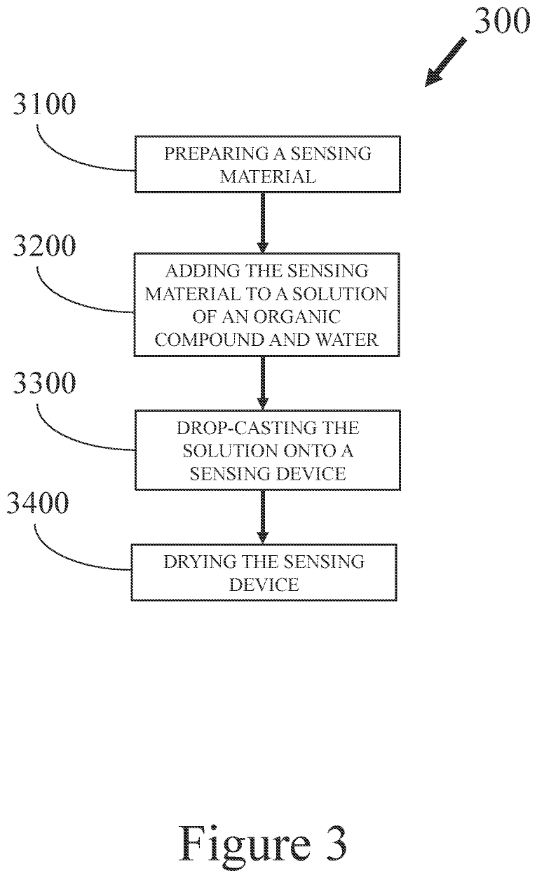

As shown in FIG. 3, the example detection device 10 may be prepared by a method 300. As shown in FIG. 3, the method 300 may include: a preparing step 3100, an adding step 3200, a drop-casting step 3300, and a drying step 3400. In some examples, the preparing step 3100 includes preparing a sensing material. In this example, the preparing step 3100 is the method 200 of FIG. 2 and the sensing material is the sensing material 212 discussed herein.

The example adding step 3200 includes adding the sensing material to a solution of an organic compound and water. In one example, the organic compound is dimethylformamide and the ratio of water: dimethylformamide is 3:1.

In addition, the example drop-casting step 3300 includes drop-casting the solution of step 3200 onto a sensing device. In some examples, the sensing device is the detection device 10 discussed herein and the detection device 10 may be fabricated, in whole or in part, on a micro hot-plate, printed circuit board (PCB) or flexible polyimide substrate. The sensor array 200 may contain one or more individually addressable sensors 200, as shown in FIG. 1. In some examples, these sensors are electrical gas sensors such as chemiresistors. In some examples, the sensor comprises a Micro-Electro-Mechanical System (MEMS) micro hot plate. The detection device 10 may also include a gas sensor housing configured to cover the sensor array 200 and employing an air burn-in process to remove any residues and fabricate phyllosilicate derivates. When utilized, the air burn-in process may be performed at any suitable temperature, including for instance at a temperature of 300° C. with a continuous air purge. Once the example drop-casting step 3300 is completed, the drying step 3400 includes drying the sensing device, such as drying at ambient conditions or 100 °C.

It has been determined that the sensing performance of the sensing material 212 depends on its temperature. For example, the sensing material 212 may react more intensely (e.g., undergo a greater change in electrical properties) to a particular analyte at a higher temperature than at a lower temperature. Accordingly, in one example, each sensor 200 contains an optional heating element 220 which is used to adjust the sensing performance of the sensing material 212. The example heating element 220 may be any suitable heating element configured to generate heat and to provide (e.g., direct, aim, guide, broadcast, etc.) that generated heat to the sensing material 212. For example, the heating element 220 may be a microheater made of platinum, gold, silver, nichrome, nickel, tungsten, titanium, aluminum, copper, graphene, carbon nanotubes, or other suitable material. In some examples, the heating element 220 is made of metal alloys such as titanium nitride, gallium nitride, gallium arsenide, Dilver P1 (an alloy of nickel, cobalt, and iron), polysilicon, or any other suitable metal alloy. Although reference is made to the heating element 220 as a single component, this disclosure should not be read as limited to the heating element 220 being a single heating element, such that this disclosure includes the heating element 220 being made of or including multiple heating elements (e.g., a heater with multiple coils, etc.).

It has been determined that the sensing performance of the sensing material 212 depends on the intensity and wavelengths of the light incident upon the sensing material 212. For example, the sensing material 212 may react more intensely (e.g., undergo a greater change in electrical properties) to a particular analyte when exposed to light with a shorter wavelength than when exposed to light with a longer wavelength. Accordingly, in one example, each sensor 200 contains an optional lighting element 230 which is used to adjust the sensing performance of the sensing material 212. The example lighting element 230 may be any suitable lighting element configured to generate light with various wavelengths and intensity and to provide (e.g., direct, aim, guide, broadcast, shine, etc.) that generated light onto the sensing material 212. For example, the lighting element 230 may be one or more light-emitting diodes (LEDs) and/or diode lasers, although other suitable lighting elements may be utilized. Although reference is made to the lighting element 230 as a single component, this disclosure should not be read as limited to a single lighting element but should be read as including a lighting element having multiple lighting elements (e.g., an array with multiple LEDs).

By adjusting the temperature of the sensing material 212, the heating element 220 may adjust the sensing characteristics of the sensing material 212. For example, in some cases the heating element 220 may influence the temperature of the sensing material 212 to increase the sensitivity of the sensing material 212 toward a specific target analyte and/or to increase the response and recovery time by which the sensing material 212 responds to that analyte. Conversely, in other cases, the heating element 220 may influence the temperature of the sensing material 212 to decrease the sensitivity of the sensing material 212 toward a specific analyte and/or to decrease the response/recovery time by which the sensing material 212 responds to that analyte.

By adjusting the intensity and/or wavelengths of light incident on the sensing material 212, the lighting element 230 adjusts the sensing characteristics of the sensing material 212. For example, the lighting element 230 may adjust the intensity and/or wavelengths of the light incident of the sensing material 212 to increase the sensitivity of the sensing material 212 toward a specific analyte and/or to increase the speed by which the sensing material 212 responds to that analyte. Conversely, in other cases, the lighting element 230 may adjust the intensity and/or wavelengths of the light incident of the sensing material 212 to decrease the sensitivity of the sensing material 212 toward a specific analyte and/or to decrease the speed by which the sensing material 212 responds to that analyte.

In some examples, the heating element 220 may be a single heating element such that one or more sensors 200 share a single heating element. Likewise, the lighting element 230 may be a single lighting element such that one or more sensors 200 share a single lighting element. The various sensors 200 comprising the sensor array 100 may contain the same or different sensing elements 210. In this way, in some examples, the sensor array 100 may contain a plurality of different sensing elements 210. Likewise, the various sensors 200 comprising the sensor array 100 may each employ different types of heating elements 220. For example, some of the sensors 200 may employ micro hotplate made of one material, while other sensors may employ micro hotplate made of another material. Similarly, the various sensors 200 comprising the sensor array 100 may each employ different types of the lighting elements 230. For example, some of the sensors 200 may employ lighting elements that include LEDs, while other sensors employ lighting elements that include lasers.

Referring once again to FIG. 1 and as disclosed in U.S. application Ser. No. 63/268,814, the controller 300 of the detection device 10 may include a readout unit 310, a heating control unit 320, a lighting control unit 330, an analysis unit 340, and a communication unit 350. The readout unit 310, the heating control unit 320, the lighting control unit 330, the analysis unit 340, and the communication unit 350 may each execute computer-executable instructions stored in a memory. The readout unit 310, the heating control unit 320, the lighting control unit 330, the analysis unit 340, and the communication unit 350 may comprise multiple separate devices or a single device such as a single microcontroller. Additionally, the controller 300 may be integrated in whole or in part within the detection device 10 or may comprise a separate device that communicates, for example, via a wired or wireless connection, with the detection device. In some examples, the controller 300 is electronically coupled to an external component (e.g., a computer or processor that executes computer-executable instructions stored in a memory).

The readout unit 310 may measure the electrical properties of each sensing material 212. For example, the readout unit may measure the resistance, capacitance, and/or impedance of the sensing material 212 by measuring the resistance, capacitance, and/or impedance between its electrode pair 211. The readout unit 310 may make a single measurement or a sequence of measurements so as to record the variation in resistance, capacitance, and/or impedance over time. The readout unit 310 may store the measurements in computer readable media for processing by the analysis unit 340.

The heating control unit 320 may control the temperature of the heating elements 220. The heating control unit 320 may provide commands to each individual heating element 220 causing that heating element 220 to maintain the temperature of the corresponding sensing material 212 according to a temperature profile. The temperature profile characterizes the desired variation in the temperature of the sensing material 212 during the period of sensing. The heating control unit 320 contains (e.g., in computer readable media) stored temperature profiles for each different sensing material 212 and for each target analyte. In this way, the heating control unit 320 controls the temperature of each sensing material 212 so as to achieve conditions suited for detecting the target analyte or analytes. These temperature profiles may be generated according to an iterative process that leverages machine learning, as described in application 63/268,814.

The lighting control unit 330 may control the illumination produced by the lighting elements 230. The lighting control unit 330 may provide commands to each individual lighting element 230 causing that lighting element 230 to illuminate the corresponding material 212 according to an illumination profile. The illumination profile may characterize the desired variation in wavelength composition and intensity of light incident on the sensing material 212 during the period of sensing. The lighting control unit 330 may contain (e.g., in computer readable media) illumination profiles for each different sensing material 212 and each target analyte. In this way, the lighting control unit 330 may control the incident light on each sensing material 212 so as to achieve conditions suited for detecting the target analyte or analytes. These illumination profiles may be generated according to an iterative process that leverages machine learning, as described in application 63/268,814.

The analysis unit 340 processes the measurement data from the readout unit 310 to identify the presence or absence of target analytes. In some embodiments, the analysis unit 340 employs a machine learning model that receives as input the measurement data from the readout unit 310 and produces as output an identity and/or concentration of the detected analytes. By considering the electrical responses of multiple sensors 200 comprising the array 100, this machine learning model can achieve higher accuracy at determining the identity and/or concentration of analytes than is typically possible by considering the electrical response of only a single sensor.

The communication unit 350 may provide a user interface for the user of the detection device 10 to operate it, including specifying the target analytes to be detected. The communication unit 350 may also display the results of the detection such as the presence and absence of target analytes and the concentrations of the analytes that are present. The communication unit 350 may also communicate with other devices such as mobile phones, desktop computers, cloud computers, etc. so as to enable the detection device 10 to be operated remotely and/or to transmit detection results.

In some examples, the detection device 10 may be fabricated, in whole or in part, on a printed circuit board (PCB) or flexible polyimide substrate. The sensor array 100 may contain one or more individually addressable sensors 200. In some examples, these sensors are electrical gas sensors. In some examples, a sensor comprises a Micro-Electro-Mechanical System (MEMS) micro hotplates. Each electrical gas sensor may include the sensing element 210, the heating element 220, and the lighting element 230. The detection device 10 may include the controller 300 such that the controller 300 is configured to adjust the conditions (e.g., temperature) for the sensing material 212 of each sensing element 210. The sensing material 212 may comprise a semiconducting sensing material that electrically bridges an electrode pair 211. In some examples, the electrode pair 211 is a pair of source-drain electrodes. In some examples, the controller 300 may be fabricated on a PCB while the sensor array 100 is fabricated on a flexible polyimide substrate. In some examples, the detection device 10 includes a sensor housing. In some examples, the sensor housing is a gas flow chamber.

In some examples, the heating of the sensing material 212 by the heating element 220 and/or the illumination of the sensing material 212 by the lighting element 230 may facilitate detection of the target analyte by inducing oxidation and/or reduction of that analyte. In some examples, this heating and/or illumination of the sensing material 212 may facilitate detection of the target analyte by inducing chemical and/or physical changes in the absorption and/or desorption properties of the sensing material 212. In some examples, this heating and/or illumination of the sensing material 212 may facilitate detection of the target analyte by altering the baseline electrical properties (e.g., the Fermi level, grain boundary potential barrier, work function, dielectric constant, etc.) of the sensing material 212. In some examples, this heating and/or illumination of the sensing material 212 may facilitate detection of the target analyte by altering the surface reactivity of the sensing material 212. In some examples, the illumination of the sensing material 212 by the lighting element 230 may alter the amount of photogenerated free electron-hole pairs in the sensing material 212, thus facilitating the detection of the target analyte.

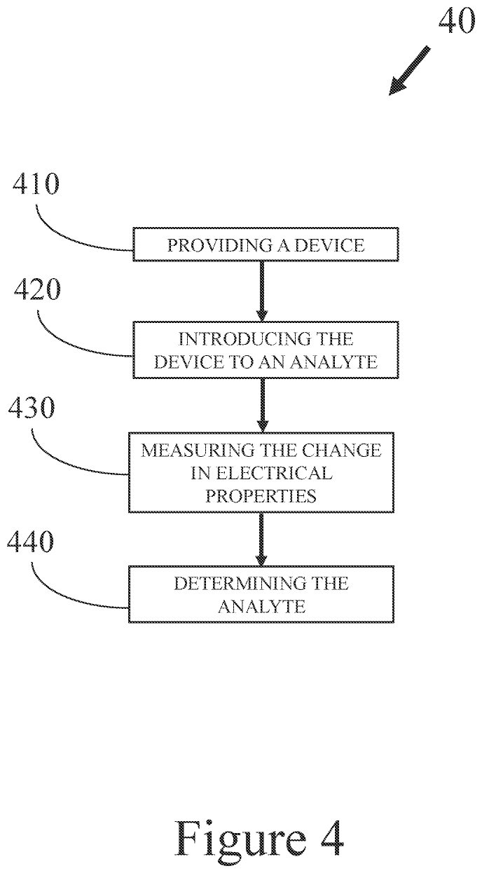

FIG. 4 is a is a flow chart illustrating an example method 40 of detecting target analytes. The method 40 may be performed, in whole or in part, by the detection device 10 and, in particular, the sensor 200. In a step 410, a device is provided, such as the detection device 10 disclosed herein. In a step 420, the device is introduced to a target analyte. In some examples, the device is exposed to a target analyte, which may be an inorganic gas, a volatile organic compound, or any combinations thereof. In some examples, the inorganic gas is ammonia, nitrogen dioxide, hydrogen sulfide or carbon monoxide, while in some examples, the volatile organic compound is toluene, benzene, ethanol, or acetone.

As previously disclosed, in some examples, the detection device 10 is exposed to the analyte under a pre-determined temperature and where included, the optional heating element 220 adjusts the temperature. The heating element 220 may influence the temperature of the sensing material 212 to increase the sensitivity and selectivity of the sensing material 212 toward a specific target analyte and/or to increase the response/recovery time by which the sensing material 212 responds to that target analyte. Conversely, in other cases, the heating element 220 may influence the temperature of the sensing material 212 to decrease the sensitivity of the sensing material 212 toward a specific target analyte and/or to decrease the response/recovery time by which the sensing material 212 responds to that analyte.

Similarly, in some examples, the detection device 10 is exposed to the analyte under a pre-determined intensity and/or wavelength of light and where included, the optional lighting element 230 adjusts the intensity and/or wavelength of light. The lighting element 230 may adjust the intensity and/or wavelength of light incident on the sensing material 212 to increase the sensitivity and selectivity of the sensing material 212 toward a specific target analyte and/or to increase the response/recovery time by which the sensing material 212 responds to that target analyte. Conversely, in other cases, the lighting element 230 may adjust the intensity and/or wavelength of light on the sensing material 212 to decrease the sensitivity of the sensing material 212 toward a specific target analyte and/or to decrease the response/recovery time by which the sensing material 212 responds to that analyte.

In a step 430, a change in electrical properties is measured. The readout unit 310 of the controller 300 may measure the electrical properties of each of the sensing material 212 such as the resistance, capacitance, and/or impedance of the sensing material 212 by measuring the resistance, capacitance, and/or impedance between its electrode pair 211. The readout unit 310 may make a single measurement or a sequence of measurements so as to record the variation in resistance, capacitance, and/or impedance over time.

In a step 440, the target analyte is determined. The target analyte may be determined based on the variation in resistance, capacitance, and/or impedance of the sensing material 212.

While this disclosure has described certain examples, it will be understood that the claims are not intended to be limited to these examples except as explicitly recited in the claims. On the contrary, the instant disclosure is intended to cover alternatives, modifications and equivalents, which may be included within the spirit and scope of the disclosure. Furthermore, in the detailed description of the present disclosure, numerous specific details are set forth in order to provide a thorough understanding of the disclosed examples. However, it will be obvious to one of ordinary skill in the art that systems and methods consistent with this disclosure may be practiced without these specific details. In other instances, well known methods, procedures, components, and circuits have not been described in detail as not to unnecessarily obscure various aspects of the present disclosure.

What is claimed is:

1. A gas sensor device, comprising:
a sensor array having a plurality of sensors, each of the plurality of sensors comprising:
a sensing element, comprising:
an electrode pair; and
a sensing material electrically coupled to the electrode pair, the sensing material comprising a phyllosilicate and configured to detect a presence of a target analyte such that an electrical property of the sensing material changes in response to detection of the target analyte by the sensing material;
a heating element electrically coupled to the sensing element; and
a lighting element electrically coupled to the sensing element; and
a controller, wherein the controller is communicatively coupled to the sensor array and configured to adjust the heating element to cause the heating element to influence a temperature of the sensing element and configured to adjust the lighting element to cause the lighting element to influence an illumination of the sensing element.

2. The gas sensor device of claim 1, wherein the electrical property is resistance.

3. The gas sensor device of claim 1, wherein the heating element comprises a plurality of heating elements and the lighting element comprises a plurality of lighting elements, such that each of the plurality of sensors comprises a respective heating element of the plurality of heating elements and a respective lighting element of the plurality of lighting elements.

4. The gas sensor device of claim 1, wherein the target analyte is at least one of an inorganic gas, a volatile organic compound, or any combinations thereof.

5. The gas sensor device of claim 4, wherein the inorganic gas is ammonia, nitric oxide, nitrogen dioxide, hydrogen sulfide or carbon monoxide.

6. The gas sensor device of claim 4, wherein the volatile organic compound is toluene or benzene, ethanol, or acetone.

7. The gas sensor device of claim 1, wherein the phyllosilicate comprises a transition metal.

8. The gas sensor device of claim 7, wherein the transition metal comprises at least one of copper, cobalt, iron, nickel, manganese, zinc, tin, tungsten or any combinations thereof.

9. The gas sensor device of claim 1, wherein the sensing material is prepared by:
adding an acid and a transition metal salt to distilled water to create a first solution;
adding a base to the first solution to create a gelled solution;
heating the gelled solution to create a powder; and
separating the powder.

10. The gas sensor device of claim 9, wherein the sensing material is a nanomaterial.

11. The gas sensor device of claim 9, wherein the acid is silicic acid.

12. The gas sensor device of claim 9, wherein the transition metal salt is a nitrate.

13. The gas sensor device of claim 9, wherein the base is sodium hydroxide.

14. The gas sensor device of claim 9, wherein the heating occurs at a temperature in a range of from 150° C. to 250° C.

15. The gas sensor device of claim 9, wherein the separating comprises at least one of centrifugation, washing, drying, or any combination thereof.

16. The gas sensor device of claim 15, wherein the drying occurs at a temperature in a range of from 60° C. to 100° C.

17. A method of preparing a gas sensor device, comprising:
preparing a sensing material;
adding the sensing material to a solution of an organic compound and water;
drop-casting the solution including the sensing material onto a sensing device; and
drying the sensing device.

18. The method of claim 17, wherein the organic compound is dimethylformamide.

19. The method of claim 17, wherein the gas sensor device comprises:
a sensor array comprising a plurality of sensors, the plurality of sensors comprising:
a sensing element, comprising:
an electrode pair;
a sensing material electrically coupled to the electrode pair, the sensing material comprising at least one of a phyllosilicate, phyllosilicate derivatives, or combinations thereof, and configured to detect a presence of a target analyte such that an electrical property of the sensing material changes in response to detection of the target analyte by the sensing material; and
a heating element electrically coupled to the sensing element; and
a lighting element electrically coupled to the sensing element; and
a controller, wherein the controller is communicatively coupled to the sensor array and configured to adjust the heating element and configured to adjust the lighting element.

20. The method of claim 19, wherein the gas sensor device further comprises:
a micro-electrical-mechanical system coupled to the sensing element and integrated into a printed circuit board; and
a gas sensor housing configured to cover the sensor array.
